(19) United States
(12) Patent Application Publication
(10) Pub. No.: US 2004/0077624 A1
(43) Pub. Date: Apr. 22, 2004

(54) Novel combination

(75) Inventors: David Nathan Abraham Fox, Sandwich (GB); Bernadette Hughes, Sandwich (GB)

Correspondence Address: PFIZER INC, 150 EAST 42ND STREET, 5TH FLOOR - STOP 49, NEW YORK, NY, 10017-5612, US

(73) Assignee: Pfizer Inc.

(21) Appl. No.: 10/443,462

(22) Filed: May 22, 2003

Related U.S. Application Data
(60) Provisional application No. 60/393,418, filed on Jul. 2, 2002.
(60) Provisional application No. 60/440,206, filed on Jan. 14, 2003.

(30) Foreign Application Priority Data
May 23, 2002 (GB) 0211919.6
Dec. 20, 2002 (GB) 0229784.4

(51) Int. Cl. A61K 31/535; A61K 31/53; A61K 31/519

(52) U.S. Cl. 514/210.21; 514/243; 514/251; 514/262.1; 514/250; 514/252.16; 514/423

(57) ABSTRACT

Combinations comprising a) an inhibitor of cyclic guanosine monophosphate (cGMP) specific phosphodiesterase type 5 (PDE5) inhibitor and b) an inhibitor of angiotensin converting enzyme (ACE) are useful for treating hypertension.

The invention relates to the use of a combination of a) an inhibitor of cyclic guanosine monophosphate (cGMP) specific phosphodiesterase type 5 (PDE5) and b) an inhibitor of angiotensin converting enzyme (ACE) for treating cardiovascular and metabolic diseases, particularly hypertension.

Blood pressure (BP) is defined by a number of haemodynamic parameters taken either in isolation or in combination. Systolic blood pressure (SBP) is the peak arterial pressure attained as the heart contracts. Diastolic blood pressure is the minimum arterial pressure attained as the heart relaxes. The difference between the SBP and the DBP is defined as the pulse pressure (PP).

Hypertension, or elevated BP, has been defined as a SBP of at least 140 mmHg and/or a DBP of at least 90 mmHg. By this definition, the prevalence of hypertension in developed countries is about 20% of the adult population, rising to about 60-70% of those aged 60 or more, although a significant fraction of these hypertensive subjects have normal BP when this is measured in a non-clinical setting. Some 60% of this older hypertensive population have isolated systolic hypertension (ISH), i.e. they have an elevated SBP and a normal DBP. Hypertension is associated with an increased risk of stroke, myocardial infarction, atrial fibrillation, heart failure, peripheral vascular disease and renal impairment (Fagard, R H; Am. J. Geriatric Cardiology 11(1), 23-28, 2002; Brown, M J and Haycock, S; Drugs 59(Suppl 2), 1-12, 2000).

The pathophysiology of hypertension is the subject of continuing debate. While it is generally agreed that hypertension is the result of an imbalance between cardiac output and peripheral vascular resistance, and that most hypertensive subjects have abnormal cardiac output and increased peripheral resistance, there is uncertainty which parameter changes first (Beevers, G etal.; BMJ 322, 912-916, 2001).

Despite the large number of drugs available in various pharmacological categories, including diuretics, alpha-adrenergic antagonists, beta-adrenergic antagonists, calcium channel blockers, angiotensin converting enzyme inhibitors and angiotensin receptor antagonists, the need for effective treatments of hypertension is still not satisfied.

ACE inhibitors, which block the vasoconstrictive action of the renin-angiotensin-aldosterone system, are recommended as a first-line therapy for hypertension. They are efficacious and generally considered to be well tolerated. The most common side effect, reported by 10-20% of patients, is coughing. Other less frequently reported side effects include rash, angioedema, hyperkalemia and functional renal failure.

Phosphodiesterase type 5 is a cyclic guanosine monophosphate-specific phosphodiesterase. Inhibitors of PDE5 decrease the rate of hydrolysis of cGMP and so potentiate the actions of nitric oxide. They have been found to be useful in the treatment of male erectile dysfunction.

According to a first aspect, the present invention provides the use of a combination comprising a) a PDE5 inhibitor and b) an ACE inhibitor in the manufacture of a medicament for treating diseases, particularly cardiovascular and metabolic diseases, more particularly hypertension.

As used herein, the terms “treating” and “treatment” include palliative, curative and prophylactic treatment. The term “hypertension” includes all diseases characterised by supranormal blood pressure, such as essential hypertension, pulmonary hypertension, secondary hypertension, isolated systolic hypertension, hypertension associated with diabetes, hypertension associated with atherosclerosis, and renovascular hypertension, and further extends to conditions for which elevated blood pressure is a known risk factor. Accordingly, the term “treatment of hypertension” includes the treatment or prevention of complications arising from hypertension, and other associated co-morbidities, including congestive heart failure, angina, stroke, glaucoma and impaired renal function, including renal failure. Metabolic diseases include in particular metabolic syndrome (also known as syndrome X), diabetes and impaired glucose tolerance, including complications thereof, such as diabetic retinopathy and diabetic neuropathy.

Hereinafter combinations of a PDE5 inhibitor and an ACE inhibitor, including combinations of specific PDE5 inhibitors and specific ACE inhibitors, will be referred to as combinations of the invention.

The combinations of the invention have the advantage that they are more efficacious, more potent, less toxic or have other more desirable properties than PDE5 inhibitors or ACE inhibitors when used alone for treating hypertension.

Hereinafter the term “the PDE5 inhibitor” means a PDE5 inhibitor for use in the invention, including all pharmaceutically acceptable salts, solvates and polymorphs of that PDE5 inhibitor. Similarly, the term “the ACE inhibitor” means an ACE inhibitor for use in the invention, including all pharmaceutically acceptable salts, solvates and polymorphs of that ACE inhibitor.

The suitability of the PDE5 inhibitor and the ACE inhibitor can be readily determined by evaluation of their potency and selectivity using literature methods followed by evaluation of their toxicity, pharmacokinetics (absorption, metabolism, distribution and elimination), etc in accordance with standard pharmaceutical practice. Suitable compounds are those that are potent and selective, have no significant toxic effect at the therapeutic dose, and preferably are bioavailable following oral administration.

Potency can be defined as an IC₅₀ value, being the concentration of compound necessary to inhibit the enzyme activity by 50%. IC₅₀ values for the PDE5 inhibitors may be determined using the PDE5 assay described hereinafter. Preferably, the PDE5 inhibitors have an IC₅₀ against the PDE5 enzyme of less than 100 nM, more preferably less than 50 nM.

Selectivity ratios may readily be determined by the skilled person, by ratio of corresponding IC₅₀ values for the particular enzymes concerned. IC₅₀ values for the PDE3 and PDE4 enzyme may be determined using established literature methodology, see Ballard S A et al.; Journal of Urology 159, 2164-2171, 1998.

Preferably the PDE5 inhibitors are selective for the PDE5 enzyme. Preferably they have a selectivity for PDE5 over PDE3 of greater than 100, more preferably greater than 300. More preferably the PDE5 has a selectivity over both PDE3 and PDE4 of greater than 100, more preferably greater than 300.

Preferably the PDE5 inhibitors have an IC₅₀ against PDE5 of less than 100 nM and a selectivity over PDE3 of greater than 100 fold.

Oral bioavailability refers to the proportion of an orally administered drug that reaches the systemic circulation. The factors that determine oral bioavailability of a drug are dissolution, membrane permeability and hepatic clearance. Typically, a screening cascade of firstly in vitro and then in vivo techniques is used to determine oral bioavailability.

Dissolution, the solubilisation of the drug by the aqueous contents of the gastro-intestinal tract (GIT), can be predicted from in vitro solubility experiments conducted at appropriate pH to mimic the GIT. Preferably the PDE5 inhibitors have a minimum solubility of 50 μg/ml. Solubility can be determined by standard procedures known in the art such as described in Lipinski C A et al.; Adv. Drug Deliv. Rev. 23(1-3), 3-25, 1997.

Membrane permeability refers to the passage of a compound through the cells of the GIT. Lipophilicity is a key property in predicting this and is determined by in vitro Log D_{7.4} measurements using organic solvents and buffer. Preferably the PDE5 inhibitors have a Log D_{7.4} of −2 to +4, more preferably −1 to +3. The Log D can be determined by standard procedures known in the art such as described in Stopher, D and McClean, S; J. Pharm. Pharmacol. 42(2), 144, 1990.

Cell monolayer assays such as Caco2 add substantially to prediction of favourable membrane permeability in the presence of efflux transporters such as P-glycoprotein, so-called Caco2 flux. Preferably, the PDE5 inhibitors have a Caco2 flux of greater than 2×10⁻⁶ cm.s⁻¹, more preferably greater than 5×10⁻⁶ cm.s⁻¹. The Caco2 flux value can be determined by standard procedures known in the art such as described in Artursson, P and Magnusson, C; J. Pharm. Sci, 79(7), 595-600, 1990.

Metabolic stability addresses the ability of the GIT to metabolise compounds during the absorption process or the liver to do so immediately post-absorption: the first pass effect. Assay systems such as microsomes, hepatocytes etc are predictive of metabolic lability. Preferably the PDE5 inhibitors show metabolic stability in the assay system that is commensurate with an hepatic extraction of less then 0.5. Examples of assay systems and data manipulation are described in Obach, R S; Curr. Opin. Drug Disc. Devel. 4(1), 36-44, 2001 and Shibata, Y et al.; Drug Met. Disp. 28(12), 1518-1523, 2000.

Because of the interplay of the above processes, further support that a drug will be orally bioavailable in humans can be gained by in vivo experiments in animals. Absolute bioavailability is determined in these studies by administering the compound separately or in mixtures by the oral route. For absolute determinations (% orally bioavailable) the intravenous route is also employed. Examples of the assessment of oral bioavailability in animals can be found in Ward, K W et al.; Drug Met. Disp. 29(1), 82-87, 2001; Berman, J et al.; J. Med. Chem. 40(6), 827-829, 1997 and Han K S and Lee, M G; Drug Met. Disp. 27(2), 221-226, 1999.

Examples of PDE5 inhibitors for use with the invention are:

The pyrazolo[4,3-d]pyrimidin-7-ones disclosed in EP-A-0463756, EP-A-0526004 and published international patent applications WO 93/06104, WO 98/49166, WO 99/54333, WO 00/24745, WO 01/27112 and WO 01/27113; the pyrazolo[3,4-d]pyrimidin-4-ones disclosed in EP-A-0995750, EP-A-0995751 and published international patent application WO 93/07149; the pyrazolo[4,3-d]pyrimidines disclosed in published international patent applications WO 01/18004, WO 02/00660 and WO 02/59126; the quinazolin-4-ones disclosed in published international patent application WO 93/12095; the pyrido[3,2-d]pyrimidin-4-ones disclosed in published international patent application WO 94/05661; the purin-6-ones disclosed in EP-A-1092718 and in published international patent application WO 94/00453; the hexahydro-pyrazino[2′,1′:6,1]pyrido[3,4-b]indole-1,4-diones disclosed in published international application WO 95/19978; the imidazo[5,1-f][1,2,4]triazin-ones disclosed in EP-A-1092719 and in published international application WO 99/24433; the bicyclic compounds disclosed in published international application WO 93/07124 and the imidazoquinazolinones disclosed in Rotella D P et al; J. Med. Chem. 43(7), 1257-1263, 2000.

The contents of the published patent applications and journal articles and in particular the general formulae of the therapeutically active compounds of the claims and exemplified compounds therein are incorporated herein in their entirety by reference thereto.

Still further examples of PDE5 inhibitors for use with the invention include: 4-bromo-5-(pyridylmethylamino)-6-[3-(4-chlorophenyl)-propoxy]-3(2H)pyridazinone; 1-[4-[(1,3-benzodioxol-5-ylmethyl)amino]-6-chloro-2-quinozolinyl]-4-piperidine-carboxylic acid, monosodium salt; (+)-cis-5,6a,7,9,9,9a-hexahydro-2-[4-(trifluoromethyl)-phenylmethyl-5-methyl-cyclopent-4,5]imidazo[2,1-b]purin-4(3H)one; furazlocillin; cis-2-hexyl-5-methyl-3,4,5,6a,7,8,9,9a- octahydrocyclopent[4,5]-imidazo[2,1-b]purin-4-one; 3-acetyl-1-(2-chlorobenzyl)-2-propylindole-6-carboxylate; 3-acetyl-1-(2-chlorobenzyl)-2-propylindole-6-carboxylate; 4-bromo-5-(3-pyridylmethylamino)-6-(3-(4-chlorophenyl)propoxy)-3-(2H)pyridazinone; I-methyl-5-(5-morpholinoacetyl-2-n-propoxyphenyl)-3-n-propyl-1,6-dihydro-7H-pyrazolo(4,3-d)pyrimidin-7-one; 1-[4-[(1,3-benzodioxol-5-ylmethyl)amino]-6-chloro-2-quinazolinyl]-4-piperidinecarboxylic acid, monosodium salt; Pharmaprojects No. 4516 (Glaxo Wellcome); Pharmaprojects No. 5051 (Bayer); Pharmaprojects No. 5064 (Kyowa Hakko; see WO 96/26940); Pharmaprojects No. 5069 (Schering Plough); GF-196960 (Glaxo Wellcome); E-8010 and E-4010 (Eisai); Bay-38-3045 & 38-9456 (Bayer) and Sch-51866.

Preferred PDE5 inhibitors for use with the invention include:

5-[2-ethoxy-5-(4-methyl-1-piperazinylsulphonyl)phenyl]-1-methyl-3-n-propyl-1,6-dihydro-7H-pyrazolo[4,3-d]pyrimidin-7-one (sildenafil) also known as 1-[[3-(6,7-dihydro-1-methyl-7-oxo-3-propyl-1H-pyrazolo[4,3-d]pyrimidin-5-yl)-4-ethoxyphenyl]sulphonyl]-4-methylpiperazine (see EP-A-0463756);

5-(2-ethoxy-5-morpholinoacetylphenyl )-1-methyl-3-n-propyl-1,6-dihydro-7H-pyrazolo[4,3-d]pyrimidin-7-one (see EP-A-0526004);

3-ethyl-5-[5-(4-ethylpiperazin-1-ylsulphonyl)-2-n-propoxyphenyl]-2-(pyridin-2-yl)methyl-2,6-dihydro-7H-pyrazolo[4,3-d]pyrimidin-7-one (see WO98/49166);

3-ethyl-5-[5-(4-ethylpiperazin-1-ylsulphonyl)-2-(2-methoxyethoxy)pyridin-3-yl]-2-(pyridin-2-yl)methyl-2,6-dihydro-7H-pyrazolo[4,3-d]pyrimidin-7-one (see WO99/54333);

(+)-3-ethyl-5-[5-(4-ethylpiperazin-1-ylsulphonyl)-2-(2-methoxy-1(R)-methylethoxy)pyridin-3-y]-2-methyl-2,6-dihydro-7H-pyrazolo[4,3-d]pyrimidin-7-one, also known as 3-ethyl-5-{5-[4-ethylpiperazin-1-ylsulphonyl]-2-([(1R)-2-methoxy-1-methylethyl]oxy)pyridin-3-yl}-2-methyl-2,6-dihydro-7H-pyrazolo[4,3-d]pyrimidin-7-one (see WO99/54333);

5-[2-ethoxy-5-(4-ethylpiperazin- 1-ylsulphonyl)pyridin-3-yl]-3-ethyl-2-[2-methoxyethyl]-2,6-dihydro-7H-pyrazolo[4,3-d]pyrimidin-7-one, also known as 1-{6-ethoxy-5-[3-ethyl-6,7-dihydro-2-(2-methoxyethyl)-7-oxo-2H-pyrazolo[4,3-d]pyrimidin-5-yl]-3-pyridylsulphonyl}-4-ethylpiperazine (see WO 01/27113, Example 8);

5-[2-iso-butoxy-5-(4-ethylpiperazin-1-ylsulphonyl)pyridin-3-yl]-3-ethyl-2-(1-methylpiperidin-4-yl)-2,6-dihydro-7H-pyrazolo[4,3-d]pyrimidin-7-one (see WO 01/27113, Example 15);

5-[2-ethoxy-5-(4-ethylpiperazin-1-ylsulphonyl)pyridin-3-yl]-3-ethyl-2-phenyl-2,6-dihydro-7H-pyrazolo[4,3-d]pyrimidin-7-one (see WO 01/27113, Example 66);

5-(5-acetyl-2-propoxy-3-pyridinyl)-3-ethyl-2-(1-isopropyl-3-azetidinyl)-2,6-dihydro-7H-pyrazolo[4,3-d]pyrimidin-7-one (see WO 01/27112, Example 124);

5-(5-acetyl-2-butoxy-3-pyridinyl)-3-ethyl-2-(1-ethyl-3-azetidinyl)-2,6-dihydro-7H-pyrazolo[4,3-d]pyrimidin-7-one (see WO 01/27112, Example 132);

(6R,12aR)-2,3,6,7,12,12a-hexahydro-2-methyl-6-(3,4-methylenedioxyphenyl)-pyrazino[2′,1′:6,1]pyrido[3,4-b]indole-1,4-dione (tadalafil, IC-351), i.e. the compound of examples 78 and 95 of published international application WO95/19978, as well as the compound of examples 1, 3, 7 and 8;

2-[2-ethoxy-5-(4-ethyl-piperazin-1-yl-1-sulphonyl)-phenyl]-5-methyl-7-propyl-3H-imidazo[5,1-f][1,2,4]triazin-4-one (vardenafil) also known as 1-[[3-(3,4-dihydro-5-methyl-3-7-propylimidazo[5,1-f]-as-triazin-2-yl)-4-ethoxyphenyl]sulphonyl]-4-ethylpiperazine, i.e. the compound of examples 20, 19, 337 and 336 of published international application WO99/24433;

[7-(3-chloro-4-methoxybenzylamino)-1-methyl-3-propyl-1H-pyrazolo[4,3-d]pyrimidin-5-ylmethoxy]acetic acid (see WO 02/59126, Example 1);

4-(4-chlorobenzyl)amino-6,7,8-trimethoxyquinazoline (Example 11 of published international application WO93/07124 (EISAI)); and

7,8-dihydro-8-oxo-6-[2-propoxyphenyl]-1H-imidazo[4,5-g]quinazoline and 1-[3-[1-[(4-fluorophenyl)methyl]-7,8-dihydro-8-oxo-1H-imidazo[4,5-g]quinazolin-6-yl]-4-propoxyphenyl]carboxamide (compounds 3 and 14 from Rotella D P et al.; J. Med. Chem. 43(7), 1257-1263, 2000).

More preferred PDE5 inhibitors for use with the invention are selected from the group and pharmaceutically acceptable salts thereof:

5-[2-ethoxy-5-(4-methyl-1-piperazinylsulphonyl)phenyl]-1-methyl-3-n-propyl-1,6-dihydro-7H-pyrazolo[4,3-d]pyrimidin-7-one (sildenafil);

(6R,12aR)-2,3,6,7,12,12a-hexahydro-2-methyl-6-(3,4-methylenedioxyphenyl) pyrazino[2′,1′:6,1]pyrido[3,4-b]indole-1,4-dione (tadalafil, IC-351);

2-[2-ethoxy-5-(4-ethyl-piperazin-1-yl-1-sulphonyl)-phenyl]-5-methyl-7-propyl-3H-imidazo[5,1-f][1,2,4]triazin-4-one (vardenafil);

5-[2-ethoxy-5-(4-ethylpiperazin-1-ylsulphonyl)pyridin-3-yl]-3-ethyl-2-[2-methoxyethyl]-2,6-dihydro-7H-pyrazolo[4,3-d]pyrimidin-7-one; and

5-(5-acetyl-2-butoxy-3-pyridinyl)-3-ethyl-2-(1-ethyl-3-azetid inyl )-2,6-dihydro-7H-pyrazolo[4,3-d]pyrimidin-7-one.

A particularly preferred PDE5 inhibitor is 5-[2-ethoxy-5-(4-methyl-1-piperazinylsulphonyl)phenyl]-1-methyl-3-n-propyl-1,6-dihydro-7H-pyrazolo[4,3-d]pyrimidin-7-one (sildenafil) (also known as 1-[[3-(6,7-dihydro-1-methyl-7-oxo-3-propyl-1H-pyrazolo[4,3-d]pyrimidin-5-yl)-4-ethoxyphenyl]sulphonyl]-4-methylpiperazine) and pharmaceutically acceptable salts thereof. Sildenafil citrate is a preferred salt.

Examples of ACE inhibitors for use with the invention include both direct-acting ACE inhibitors and prodrugs thereof, including alacepril, alindapril, altiopril, benazepril, benazeprilat, captopril, ceronapril, cilazapril, cilazaprilat, delapril, enalapril, enalaprilat, fosinopril, imidapril, indolapril, libenzapril, lisinopril, moexepril, moveltipril, pentopril, perindopril, quinapril, quinaprilat, ramipril, rentiapril, spirapril, temocapril, teprotide, trandolapril and zofenopril. Furthermore, the ACE inhibitor may be a “dual ACE/NEP inhibitor”, i.e. a compound that inhibits both ACE and neutral endopeptidase (NEP), such as, for example, omapatrilat, fasidotril, mixanpril, sampatrilat, BMS-189921, MDL-100240 and Z13752A.

Preferred combinations of PDE5 inhibitors and ACE inhibitors for treating hypertension are:

sildenafil and quinapril hydrochloride;

sidenafil and benazepril hydrochloride;

sildenafil and captopril;

sildenafil and enalapril maleate;

sildenafil and fosinopril;

sildenafil and lisinopril;

sildenafil and moexipril;

sildenafil and ramipril;

sildenafil and trandolapril;

tadalafil and quinapril hydrochloride;

tadalafil and benazepril hydrochloride;

tadalafil and captopril;

tadalafil and enalapril maleate;

tadalafil and fosinopril;

tadalafil and lisinopril;

tadalafil and moexipril;

tadalafil and ramipril;

tadalafil and trandolapril;

vardenafil and quinapril hydrochloride;

vardenafil and benazepril hydrochloride;

vardenafil and captopril;

vardenafil and enalapril maleate;

vardenafil and fosinopril;

vardenafil and lisinopril;

vardenafil and moexipril;

vardenafil and ramipril; and

vardenafil and trandolapril.

The pharmaceutical combinations of the invention are useful in the treatment of diseases including cardiovascular and metabolic diseases, and they may also be useful in the treatment of other diseases such as thrombosis, and in the management of patients following percutaneous translumenal coronary angioplasty (“post-PTCA patients”).

Preferably the cardiovascular disorder to be treated is hypertension, congestive heart failure, angina, stroke or renal failure. More preferably the cardiovascular disorder is essential hypertension, pulmonary hypertension, secondary hypertension, isolated systolic hypertension, hypertension associated with diabetes, hypertension associated with atherosclerosis, renovascular hypertension, congestive heart failure, angina, stroke or renal failure. In a particularly preferred embodiment, the disorder to be treated is essential hypertension. In another particularly preferred embodiment, the disorder to be treated is pulmonary hypertension. In another particularly preferred embodiment, the disorder to be treated is secondary hypertension. In another particularly preferred embodiment, the disorder to be treated is isolated systolic hypertension. In another particularly preferred embodiment, the disorder to be treated is hypertension associated with diabetes. In another particularly preferred embodiment, the disorder to be treated is hypertension associated with atherosclerosis. In another particularly preferred embodiment, the disorder to be treated is renovascular hypertension.

Preferably the metabolic disease to be treated is impaired glucose tolerance or diabetes, including complications thereof, such as diabetic retinopathy and diabetic neuropathy. More preferably the metabolic disease is impaired glucose tolerance, type-1 diabetes, non-insulin dependent type-2 diabetes or insulin-dependent type-2 diabetes.

The combination of the invention can be administered alone but will generally be administered in admixture with a suitable pharmaceutical excipient, diluent or carrier selected with regard to the intended route of administration and standard pharmaceutical practice.

For example, the combinations of the invention can be administered orally, buccally or sublingually in the form of tablets, capsules, multi-particulates, gels, films, ovules, elixirs, solutions or suspensions, which may contain flavouring or colouring agents, for immediate-, delayed-, modified-, sustained-, pulsed- or controlled-release applications. The combinations of the invention may also be administered as fast-dispersing or fast-dissolving dosage forms or in the form of a high energy dispersion or as coated particles. Suitable formulations may be in coated or uncoated form, as desired.

Such solid pharmaceutical compositions, for example, tablets, may contain excipients such as microcrystalline cellulose, lactose, sodium citrate, calcium carbonate, dibasic calcium phosphate, glycine and starch (preferably corn, potato or tapioca starch), disintegrants such as sodium starch glycollate, croscarmellose sodium and certain complex silicates, and granulation binders such as polyvinylpyrrolidone, hydroxypropylmethylcellulose (HPMC), hydroxypropylcellulose (HPC), sucrose, gelatin and acacia. Additionally, lubricating agents such as magnesium stearate, stearic acid, glyceryl behenate and talc may be included.

The following formulation examples are illustrative only and are not intended to limit the scope of the invention. Active ingredient means a combination of the invention.

### FORMULATION 1:

A tablet is prepared using the following ingredients:

Active ingredient (50 mg) is blended with cellulose (microcrystalline), silicon dioxide, stearic acid (fumed) and the mixture is compressed to form tablets.

### FORMULATION 2:

An intravenous formulation may be prepared by combining active ingredient (100 mg) with isotonic saline (1000 ml).

The tablets are manufactured by a standard process, for example, direct compression or a wet or dry granulation process. The tablet cores may be coated with appropriate overcoats.

Solid compositions of a similar type may also be employed as fillers in gelatin or HPMC capsules. Preferred excipients in this regard include lactose, starch, a cellulose, milk sugar or high molecular weight polyethylene glycols. For aqueous suspensions and/or elixirs, the PDE5 and ACE inhibitors may be combined with various sweetening or flavouring agents, colouring matter or dyes, with emulsifying and/or suspending agents and with diluents such as water, ethanol, propylene glycol and glycerin, and combinations thereof.

Modified release and pulsatile release dosage forms may contain excipients such as those detailed for immediate release dosage forms together with additional excipients that act as release rate modifiers, these being coated on and/or included in the body of the device. Release rate modifiers include, but are not exclusively limited to, hydroxypropylmethyl cellulose, methyl cellulose, sodium carboxymethylcellulose, ethyl cellulose, cellulose acetate, polyethylene oxide, xanthan gum, carbomer, ammonio methacrylate copolymer, hydrogenated castor oil, carnauba wax, paraffin wax, cellulose acetate phthalate, hydroxypropylmethyl cellulose phthalate, methacrylic acid copolymer and mixtures thereof. Modified release and pulsatile release dosage forms may contain one or a combination of release rate modifying excipients. Release rate modifying excipients may be present both within the dosage form i.e. within the matrix, and/or on the dosage form, i.e. upon the surface or coating.

Fast dispersing or dissolving dosage formulations (FDDFs) may contain the following ingredients: aspartame, acesulfame potassium, citric acid, croscarmellose sodium, crospovidone, diascorbic acid, ethyl acrylate, ethyl cellulose, gelatin, hydroxypropylmethyl cellulose, magnesium stearate, mannitol, methyl methacrylate, mint flavouring, polyethylene glycol, fumed silica, silicon dioxide, sodium starch glycolate, sodium stearyl fumarate, sorbitol, xylitol. The terms dispersing or dissolving as used herein to describe FDDFs are dependent upon the solubility of the drug substance used i.e. where the drug substance is insoluble a fast dispersing dosage form can be prepared and where the drug substance is soluble a fast dissolving dosage form can be prepared.

The combinations of the invention can also be administered parenterally, for example, intracavernousily, intravenously, intra-arterially, intraperitoneally, intrathecally, intraventricularly, intraurethrally, intrasternally, intracranially, intramuscularly or subcutaneously, or they may be administered by infusion or needleless injection techniques. For such parenteral administration they are best used in the form of a sterile aqueous solution which may contain other substances, for example, enough salts or glucose to make the solution isotonic with blood. The aqueous solutions should be suitably buffered (preferably to a pH of from 3 to 9), if necessary. The preparation of suitable parenteral formulations under sterile conditions is readily accomplished by standard pharmaceutical techniques well-known to those skilled in the art.

The following dosage levels and other dosage levels herein are for the average human subject having a weight range of about 65 to 70 kg. The skilled person will readily be able to determine the dosage levels required for a subject whose weight falls outside this range, such as children and the elderly.

The dosage of the combination of the invention in such formulations will depend on its potency, but can be expected to be in the range of from 1 to 500 mg of PDE5 inhibitor and 1 to 100 mg of ACE inhibitor for administration up to three times a day. A preferred dose is in the range 10 to 100 mg (e.g. 10, 25, 50 and 100 mg) of PDE5 inhibitor and 5 to 50 mg (e.g. 5, 10, 25 and 50 mg) of ACE inhibitor which can be administered once, twice or three times a day (preferably once). However the precise dose will be as determined by the prescribing physician and will depend on the age and weight of the subject and severity of the symptoms.

For oral and parenteral administration to human patients, the daily dosage level of a combination of the invention will usually be from to 5 to 500 mg/kg (in single or divided doses).

Thus tablets or capsules may contain from 5 mg to 250 mg (for example 10 to 100 mg) of the combination of the invention for administration singly or two or more at a time, as appropriate. The physician in any event will determine the actual dosage which will be most suitable for any individual patient and it will vary with the age, weight and response of the particular patient. The above dosages are exemplary of the average case. There can, of course, be individual instances where higher or lower dosage ranges are merited and such are within the scope of this invention. The skilled person will appreciate that the combinations of the invention may be taken as a single dose as needed or desired (i.e. prn). It is to be appreciated that all references herein to treatment include acute treatment (taken as required) and chronic treatment (longer term continuous treatment).

The combinations of the invention can also be administered intranasally or by inhalation and are conveniently delivered in the form of a dry powder inhaler or an aerosol spray presentation from a pressurised container, pump, spray, atomiser or nebuliser, with or without the use of a suitable propellant, e.g. dichlorodifluoromethane, trichlorofluoromethane, dichlorotetrafluoroethane, a hydrofluoroalkane such as 1,1,1,2-tetrafluoroethane (HFA 134A [trade mark]) or 1,1,1,2,3,3,3-heptafluoropropane (HFA 227EA [trade mark]), carbon dioxide or other suitable gas. In the case of a pressurised aerosol, the dosage unit may be determined by providing a valve to deliver a metered amount. The pressurised container, pump, spray, atomiser or nebuliser may contain a solution or suspension of the active compound, e.g. using a mixture of ethanol and the propellant as the solvent, which may additionally contain a lubricant, e.g. sorbitan trioleate. Capsules and cartridges (made, for example, from gelatin) for use in an inhaler or insufflator may be formulated to contain a powder mix of the combinations of the invention and a suitable powder base such as lactose or starch.

Aerosol or dry powder formulations are preferably arranged so that each metered dose or “puff” contains from 1 μg to 50 mg of a combination of the invention for delivery to the patient. The overall daily dose with an aerosol will be in the range of from 1 μg to 50 mg which may be administered in a single dose or, more usually, in divided doses throughout the day.

Alternatively, the combinations of the invention can be administered in the form of a suppository or pessary, or they may be applied topically in the form of a gel, hydrogel, lotion, solution, cream, ointment or dusting powder. The combinations of the invention may also be dermally or transdermally administered, for example, by the use of a skin patch, depot or subcutaneous injection. They may also be administered by the pulmonary or rectal routes.

For application topically to the skin, the combinations of the invention can be formulated as a suitable ointment containing the active compound suspended or dissolved in, for example, a mixture with one or more of the following: mineral oil, liquid petrolatum, white petrolatum, propylene glycol, polyoxyethylene polyoxypropylene compound, emulsifying wax and water. Alternatively, they can be formulated as a suitable lotion or cream, suspended or dissolved in, for example, a mixture of one or more of the following: mineral oil, sorbitan monostearate, a polyethylene glycol, liquid paraffin, polysorbate 60, cetyl esters wax, cetearyl alcohol, 2-octyldodecanol, benzyl alcohol and water.

The combinations of the invention may also be used in combination with a cyclodextrin. Cyclodextrins are known to form inclusion and non-inclusion complexes with drug molecules. Formation of a drug-cyclodextrin complex may modify the solubility, dissolution rate, bioavailability and/or stability property of a drug molecule. Drug-cyclodextrin complexes are generally useful for most dosage forms and administration routes. As an alternative to direct complexation with the drug the cyclodextrin may be used as an auxiliary additive, e.g. as a carrier, diluent or solubiliser. Alpha-, beta- and gamma-cyclodextrins are most commonly used and suitable examples are described in published international patent applications WO91/11172, WO94/02518 and WO98/55148.

Oral administration of the combinations of the invention is a preferred route, being the most convenient. In circumstances where the recipient suffers from a swallowing disorder or from impairment of drug absorption after oral administration, the drug may be administered parenterally, sublingually or buccally.

The combinations of the invention may be used as part of a triple therapy regimen, i.e. a treatment protocol in which the patient is treated with three pharmaceutical agents. The third agent in the triple therapy may be a second PDE5 or ACE inhibitor, or it may be chosen from a third pharmacological group. For example, it may be a neutral endopeptidase inhibitor, an angiotensin II receptor antagonist, a calcium channel blocker such as amlodipine, a statin such as atorvastatin, a beta blocker (i.e. a beta-adrenergic receptor antagonist) or a diuretic.

It will be appreciated that the invention covers the following further aspects and that the embodiments specified hereinabove for the first aspect extend to these aspects:

i) a pharmaceutical combination of the invention (for simultaneous, separate or sequential administration) for treating hypertension;

ii) a kit for treating hypertension, the kit comprising: a) a first pharmaceutical composition comprising a PDE5 inhibitor; b) a second pharmaceutical composition comprising an ACE inhibitor; and c) a container for the compositions;

iii) a method of treating hypertension in a subject comprising treating said patient with an effective amount of a combination of the invention.

Assay

Preferred compounds suitable for use in accordance with the present invention are potent and selective PDE5 inhibitors. In vitro PDE inhibitory activities against cyclic guanosine 3′,5′-monophosphate (cGMP) and cyclic adenosine 3′,5′-monophosphate (cAMP) phosphodiesterases can be determined by measurement of their IC₅₀ values (the concentration of compound required for 50% inhibition of enzyme activity).

The required PDE enzymes can be isolated from a variety of sources, including human corpus cavernosum, human and rabbit platelets, human cardiac ventricle, human skeletal muscle and bovine retina, essentially by a modification of the method of Thompson, W J et al.; Biochemistry 18(23), 5228-5237, 1979, as described by Ballard S A et al.; J. Urology 159(6), 2164-2171, 1998. In particular, cGMP-specific PDE5 and cGMP-inhibited cAMP PDE3 can be obtained from human corpus cavernosum tissue, human platelets or rabbit platelets; cGMP-stimulated PDE2 was obtained from human corpus cavernosum; calcium/calmodulin (Ca/CAM)-dependent PDE1 from human cardiac ventricle; cAMP-specific PDE4 from human skeletal muscle; and photoreceptor PDE6 from bovine retina. Phosphodiesterases 7-11 can be generated from full length human recombinant clones transfected into SF9 cells.

Assays can be performed either using a modification of the “batch” method of Thompson W J and Appleman M M; Biochemistry 10(2),311-316, 1971, essentially as described by Ballard S A et al.; J. Urology 159(6), 2164-2171, 1998, or using a scintillation proximity assay for the direct detection of [³H]-labelled AMP/GMP using a modification of the protocol described by Amersham plc under product code TRKQ7090/7100. In summary, for the scintillation proximity assay the effect of PDE inhibitors was investigated by assaying a fixed amount of enzyme in the presence of varying inhibitor concentrations and low substrate, (cGMP or cAMP in a 3:1 ratio unlabelled to [³H]-labeled at a concentration of ˜1/3 Kₘ or less) such that IC₅₀≅Kᵢ. The final assay volume was made up to 100 μl with assay buffer [20 mM Tris-HCl pH 7.4, 5 mM MgCl₂, lmg/ml bovine serum albumin]. Reactions were initiated with enzyme, incubated for 30-60 minutes at 30° C. to give <30% substrate turnover and terminated with 50 μl yttrium silicate SPA beads (containing 3 mM of the respective unlabelled cyclic nucleotide for PDEs 9 and 11). Plates were re-sealed and shaken for 20 minutes, after which the beads were allowed to settle for 30 minutes in the dark and then counted on a TopCount plate reader (Packard, Meriden, C T) Radioactivity units were converted to % activity of an uninhibited control (100%), plotted against inhibitor concentration and inhibitor IC₅₀ values obtained using the ‘Fit Curve’ Microsoft Excel extension.

Animal study

The efficacy of the combinations of the invention has been demonstrated in an animal model of human hypertension using enalapril as a representative ACE inhibitor and 3-ethyl-5-[5-(4-ethylpiperazin-1-ylsulphonyl)-2-(2-methoxyethoxy)pyridin-3-yl]-2-(pyridin-2-yl )methyl-2,6-dihydro-7H-pyrazolo[4,3-d]pyrimidin-7-one (the compound of Example 4 of published international patent application WO99/54333) as a representative PDE5 inhibitor.

Animals

The spontaneously hypertensive rat (SHR) is a widely used model of human hypertension. Male SHRs (20-22 weeks old) were instrumented with Doppler flow probes for the measurement of mesenteric, hindquarters and renal blood flow, aortic blood pressure and heart rate according to published methods (Gardiner, S M et al.; Br. J. Pharmacol. 132(8), 1625-1629, 2001).

Drugs

Solutions of enalapril (7.5 μg/mL), PDE5 inhibitor (200μg/mL) and a combination of enalapril and PDE5 inhibitor (7.5μg/mL+200 μg/mL) were infused at a rate of 0.4 mL/h throughout the experimental period. Control animals received compound vehicle; isotonic saline adjusted to pH 4 with hydrochloric acid.

Protocol

Baseline haemodynamic parameters were recorded. Animals (n=7 or 8/group) were randomised then treated with the drug solution by continuous infusion over 4 days. Changes in haemodynamic parameters were monitored during the study period for 7h on each day. Summary results expressed as difference from the vehicle response are presented in the Table below.

| | Treatment | | |
|---|---|---|---|
| | Enal- | PDE5 in- | Com- |
| | april | hibitor | bination |
| Overall change in mean BP (mmHg) | −2.4 | −12.1 | −17.8 |
| Change in mesenteric conductance (%) | +22.4 | +22.1 | +48.1 |
| Change in renal conductance (%) | +14.2 | −0.8 | +34.2 |
| Change in aortic conductance (%) | +3.7 | +19.8 | +30.1 |

The results, for renal conductance in particular, demonstrate that the two agents in combination can produce an effect that is greater than the sum of their individual effects.

1. The use of a combination of an inhibitor of cyclic guanosine monophosphate specific phosphodiesterase type 5 (PDE5) and an inhibitor of angiotensin converting enzyme (ACE) for the preparation of a medicament for the palliative, curative or prophylactic treatment of hypertension, including essential hypertension, pulmonary hypertension, secondary hypertension, isolated systolic hypertension, hypertension associated with diabetes, hypertension associated with atherosclerosis and renovascular hypertension, congestive heart failure, angina, stroke, diabetes and impaired glucose tolerance.

2. The use according to claim 1, wherein the inhibitor of PDE5 has an IC₅₀ value of less than 100 nM.

3. The use according to claim 2, wherein the inhibitor of PDE5 has an IC₅₀ value of less than 50 nM.

4. The use according to any preceding claim, wherein the inhibitor of PDE5 is selected from
5-[2-ethoxy-5-(4-methyl-1-piperazinylsulphonyl)phenyl]-1-methyl-3-n-propyl-1,6-dihydro-7H-pyrazolo[4,3-d]pyrimidin-7-one (sildenafil);
(6R,12aR)-2,3,6,7,12,12a-hexahydro-2-methyl-6-(3,4-methylenedioxyphenyl) pyrazino[2′,1′:6,1]pyrido[3,4-b]indole-1,4-dione (tadalafil);
2-[2-ethoxy-5-(4-ethyl-piperazin-1-yl-1-sulphonyl)-phenyl]-5-methyl-7-propyl-3H-imidazo[5,1-f][1 ,2,4]triazin-4-one (vardenafil);
5-[2-ethoxy-5-(4-ethylpiperazin- 1-ylsulphonyl )pyridin-3-yl]-3-ethyl-2-[2-methoxyethyl]-2,6-dihydro-7H-pyrazolo[4,3-d]pyrimidin-7-one; and
5-(5-acetyl-2-butoxy-3-pyridinyl)-3-ethyl-2-(1-ethyl-3-azetidinyl)-2 ,6-dihydro-7H-pyrazolo[4,3-d]pyrimidin-7-one
and pharmaceutically acceptable salts thereof.

5. The use according to claim 4, wherein the inhibitor of PDE5 is 5-[2-ethoxy-5-(4-methyl-1-piperazinylsulphonyl)phenyl]-1-methyl-3-n-propyl-1,6-dihydro-7H-pyrazolo[4,3-d]pyrimidin-7-one (sildenafil) or a pharmaceutically acceptable salt thereof.

6. The use according to claim 5, wherein the inhibitor of PDE5 is sildenafil citrate.

7. The use according to any preceding claim, wherein the inhibitor of ACE is selected from benazepril, captopril, cilazepril, enalapril, enalaprilat, fosinopril, lisinopril, moexepril, perindopril, quinapril, ramipril and trandolapril and pharmaceutically acceptable salts thereof.

8. The use according to claim 7, wherein the combination of the inhibitor of PDE5 and the inhibitor of ACE is selected from:
sildenafil citrate and quinapril hydrochloride;
sidenafil citrate and benazepril hydrochloride;
sildenafil citrate and captopril;
sildenafil citrate and enalapril maleate;
sildenafil citrate and fosinopril;
sildenafil citrate and lisinopril;
sildenafil citrate and moexipril;
sildenafil citrate and ramipril; and
sildenafil citrate and trandolapril

9. The use according to claim 1, wherein the medicament is for the treatment of hypertension.

10. A pharmaceutical composition comprising an inhibitor of cyclic guanosine monophosphate specific phosphodiesterase type 5 (PDE5) and an inhibitor of angiotensin converting enzyme (ACE).

11. A pharmaceutical combination for simultaneous, separate or sequential administration for treating hypertension, comprising an inhibitor of cGMP specific phosphodiesterase type 5 (PDE5) and an inhibitor of angiotensin converting enzyme (ACE).

12. A kit for treating hypertension, the kit comprising: a) a first pharmaceutical composition comprising a PDE5 inhibitor; b) a second pharmaceutical composition comprising an ACE inhibitor; and c) a container for the compositions.

13. A method of treating hypertension in a subject comprising treating said patient simultaneously, separately or sequentially with an effective amount of an inhibitor of PDE5 and an inhibitor of ACE.
